(19) United States
(12) Patent Application Publication
(10) Pub. No.: US 2004/0176720 A1
(43) Pub. Date: Sep. 9, 2004

(54) Device for administering a liquid solution of an active substance

(76) Inventor: Urs Kipfer, Luetzelflueh (CH)

Correspondence Address: David E. Bruhn, DORSEY & WHITNEY LLP, Intellectual Property Department, 50 South Sixth Street, Suite 1500, Minneaplolis, MN, 55402-1498, US

(21) Appl. No.: 10/650,473

(22) Filed: Aug. 28, 2003

Related U.S. Application Data
(60) Provisional application No. 60/407,812, filed on Sep. 3, 2002.
(60) Provisional application No. 60/407,819, filed on Sep. 3, 2002.

(30) Foreign Application Priority Data
Aug. 31, 2002 (CH) 1488/02

(51) Int. Cl. A61M 31/00

(52) U.S. Cl. 604/65

(57) ABSTRACT

The invention pertains to devices for administering a liquid solution of an active substance, wherein the subject device has at least one associated temperature sensor.

### CROSS-REFERENCE TO RELATED APPLICATION(S)

This application claims priority to Swiss Application No. 1488/02, filed on Aug. 31, 2002, and U.S. Provisional Application 60/407,812, filed on Sep. 3, 2002.

### BACKGROUND

The invention pertains to a devices and methods for administering a liquid solution of an active substance and, more particularly, to such devices and methods wherein a sensor is associated with the device.

The devices to which the invention pertains are dispensing devices for injecting or infusing a liquid solution of an active substance, preferably a medicinal liquid, e.g., insulin or growth hormones. In some embodiments, they are preferably portable types of apparatus, especially in the form of so-called injection pens and, especially preferably, infusion pumps.

### SUMMARY

Infusion pumps, for example as known from EP-B-0 143 895, for the infusion of solutions of active substances, typically have a housing in which the container for the product can be accommodated in a receptacle that is provided for this purpose. A piston is accommodated in the container in such a way that it can be displaced. When displacing the piston in the advancement direction, a dose of the product is discharged by the force of the piston. In order to do this, the container, generally an ampoule, is fixed in the receptacle in such a way that a driven element of a dispensing mechanism presses the piston in the advancement direction in order to discharge the product. The dispensing mechanism essentially comprises two elements, the aforementioned driven element and a drive device. In the case where the container has been accommodated in the housing, the driven element projects into the container toward the piston. When the drive device is actuated, it engages the driven element in such a way that the driven element can be pushed in the advancement direction. In the case of known types of pump apparatus with spindle drives that are operated by electric motors, only the drive device is actuated by rotation. Rotation, in turn, brings about advancement of the driven element, but this rotation is generally transferred directly to the piston in the case of pumping types of apparatus.

Product dispensing takes place in such a way that when actuating the dispensing mechanism, the piston is displaced over a defined path length in the direction of the container outlet and, as a result, active substance is conveyed into the infusion tube to the patient, and is conveyed into the patient via an access arrangement either in the form of a canula or a wider tube.

Such administering devices, which are frequently portable, are normally pre-programmed to a certain extent, and usually have operating knobs, memory and control units, batteries, time measurement systems, acoustic and/or vibratory signalling systems, and LCD displays. Over the last few years, the further development of these types of apparatus has concentrated on improving the dispensing accuracy, or detecting occlusions or leakages. An aspect that has been neglected in this regard is the fact that the discharge accuracy is markedly influenced by the variations in temperature to which the dispensing apparatus is subject. Insulin, for example, has almost exactly the same expansion coefficient as water. Measurements have shown that a temperature difference of +15° C. can lead to the displacement of 1 unit in a filled 300 unit container. Such a temperature difference can arise on a short-term basis, when going to bed, or in the case of being briefly exposed to the sun. Many diabetics have an average insulin delivery rate of 0.5 units per hour. Even from this example, it is found that temperature differences during the administration of insulin are very important. However, this phenomenon had not been investigated until now because the applicable standards for infusion pumps require that the dispensing accuracy be checked at a constant temperature.

The problem that the invention addresses is how to prevent or at least minimize discharging inaccuracies or false alarms by occlusion/leakage sensors of administering devices for liquid solutions of active substances as a result of temperature variations.

In one embodiment, the present invention comprises a device for administering a liquid solution of an active substance, wherein the device has at least one associated temperature sensor.

The advantages that are realized by the invention are that, essentially, dispensing inaccuracies or false alarms by occlusion/leakage sensors of administering types of apparatus for liquid solutions of active substances as a result of temperature variations can be prevented or at least minimized as a result of arranging a temperature sensor in or on the administering apparatus.

### BRIEF DESCRIPTION OR THE DRAWING

FIG. 1 depicts an administering device in accordance with the present invention.

### DETAILED DESCRIPTION

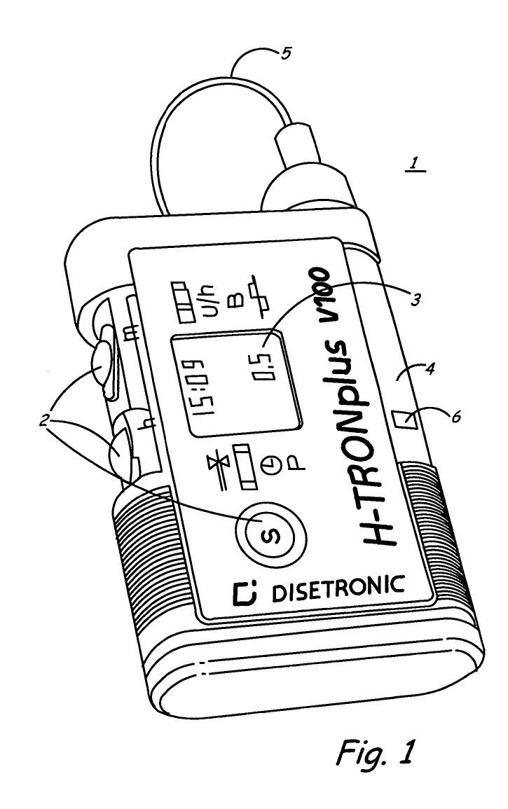

FIG. 1 shows an administering device 1 with a container 4, several operating knobs 2, an LCD display 3, portions of an infusion tube 5, and a temperature sensor 6 that is arranged near the container.

The temperature of the active substance that is to be administered can be measured extremely accurately by arranging the temperature sensor 6 in the vicinity of the container 4. An arrangement of several temperature sensors 6 and the corresponding evaluation of their signals is also conceivable in order to obtain the temperature difference of the active substance between two or more measurements with great accuracy. Naturally, the temperature sensor 6 could also be arranged in the container provided that this does not lead to contamination of the substance that is to be administered.

The measured temperature difference may be displayed on the LCD display 3, and/or it may activate an acoustic/vibratory or graphic alarm if it exceeds a stipulated difference within a defined unit of time.

In addition to or in place of such information for the user, a change is made in the pre-programmed amount that is to be dispensed, or an appropriate change in the control unit (not drawn), e.g., on the LCD display 3, is suggested to the user, where the user must acknowledge this by actuating an operating knob 2.

Occlusion or leakage sensors are based on a force or pressure measurement, which acts on the solution of the active substance or the mechanical device that discharges it, or they measure the energy that is needed by the mechanical device for the dispensing process. All these measurements are ultimately influenced by temperature differences. Threshold or alarm values for the occlusion or leakage sensors are adapted to or disconnected from the measured temperature difference via appropriate memory and control units.

While multiple embodiments are disclosed, including preferred embodiments, still other embodiments of the present invention will become apparent to those skilled in the art from the above detailed description. As will be apparent, the invention is capable of modifications in various obvious aspects, all without departing from the spirit and scope of the present invention. Accordingly, the accompanying drawing and this description are to be regarded as illustrative, not restrictive.

1. A device for administering a liquid solution of an active substance, said device comprising a temperature sensor.

2. The device in accordance with claim 1, wherein temperature differences can be measured by the temperature sensor.

3. The device in accordance with claim 2, further comprising display means whereby a measured temperature difference is displayed whenever said temperature difference exceeds a stipulated difference within a defined time.

4. The device in accordance with claim 2, further comprising an acoustic alarm activated whenever the temperature difference exceeds a stipulated difference within a defined time.

5. The device in accordance with claim 2, further comprising a vibratory alarm activated whenever the temperature difference exceeds a stipulated difference within a defined time.

6. The device in accordance with claim 3, wherein a graphic alarm displayed whenever the temperature difference exceeds a stipulated difference within a defined time.

7. The device in accordance with claim 2, wherein the pre-programmed amount to be dispensed is automatically changed whenever the temperature difference exceeds a stipulated difference within a defined time.

8. The device in accordance with claim 2, wherein an appropriate change in the pre-programmed amount is suggested to the user of the administering device whenever the temperature difference exceeds a stipulated difference within a defined time.

9. The device in accordance with claim 2, further comprising a controller and at least one of an occlusion or leakage sensor, wherein threshold or alarm values for the at least of an occlusion or leakage sensor are either appropriately adapted to or disconnected from the measured temperature difference via the controller.

10. The device in accordance with claim 9, wherein the solution of the active substance is insulin.

11. The device in accordance claim 2, wherein the device is a portable insulin pump.

12. The device in accordance with claim 2, wherein the device has more than one temperature sensor.

13. A device for the infusion of solutions of active substances, said device comprising a housing in which a container for a solution can be accommodated, a piston accommodated in the container in such a way that it can be displaced to cause the discharge of a dose of the solution from the container, a dispensing mechanism comprising a driven element for moving the piston and a drive device for moving the driven element, and a temperature sensor.

14. The device according to claim 13, further comprising a memory and control unit and inputs and outputs.

15. The device according to claim 14, wherein said outputs comprise at least one of a display and a signaling system.

16. The device in accordance with claim 15, wherein a temperature difference can be measured by the temperature sensor.

17. The device in accordance with claim 16, wherein a measured temperature difference is displayed whenever said temperature difference exceeds a stipulated difference within a defined time.

18. The device in accordance with claim 16, wherein an alarm is signaled when the temperature difference exceeds a stipulated difference within a defined time.

19. The device in accordance with claim 16, wherein a pre-programmed amount of solution to be infused is automatically changed whenever the temperature difference exceeds a stipulated difference within a defined time.

20. The device in accordance with claim 16, wherein an appropriate change in the pre-programmed amount of solution to be infused is suggested to the user of the device whenever the temperature difference exceeds a stipulated difference within a defined time.

21. The device in accordance with claim 16, further comprising at least one of an occlusion or leakage sensor, wherein threshold alarm values for the at least one of an occlusion or leakage sensor are either appropriately adapted to or disconnected from the measured temperature difference via the memory and control unit.
